SEQUENCE LISTING (5 sequences)

SEQ ID NO: 1 - protein, 535 aa, Artificial Sequence
  Modif. Diphteria toxin
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - protein, 16 aa, artificial sequence
  RhCA67-16
  MISC_FEATURE (8)..(8): Potentially hydrocarbonated on indole, such as 2,4,6-trimethoxybenzyl
FNSTIIGWIPAKPHIN

SEQ ID NO: 3 - protein, 17 aa, artificial sequence
  RhCA67-16-Cys
  MISC_FEATURE (8)..(8): Potentially hydrocarbonated on indole, such as 2,4,6-trimethoxybenzyl
FNSTIIGWIPAKPHINC

SEQ ID NO: 4 - protein, 17 aa, artificial sequence
  RhCA611
YFSRIIQKQFGHVNNGK

SEQ ID NO: 5 - protein, 18 aa, artificial sequence
  RhCA611-001-Cys
YFSRIIQKQFGHVNNGKC

(19) United States
(12) Patent Application Publication
(10) Pub. No.: US 2024/0148863 A1
(43) Pub. Date: May 9, 2024

(54) CARRIER PROTEIN FOR PEPTIDE ANTIGEN

(71) Applicants: Nicolas HAVELANGE, Bruxelles (BE); Stéphane HUBERTY, Rixensart (BE); CURAVAC EUROPE, Rixensart (BE)

(72) Inventors: Nicolas HAVELANGE, Bruxelles (BE); Stéphane HUBERTY, Rixensart (BE)

(73) Assignee: CURAVAC EUROPE S.A., Rixensart (BE)

(21) Appl. No.: 18/278,978

(22) PCT Filed: Feb. 28, 2022
(86) PCT No.: PCT/EP2022/054911

§ 371 Date: Aug. 25, 2023

(30) Foreign Application Priority Data
Feb. 26, 2021 (BE) BE2021/5141

(51) Int. Cl. A61K 39/385; C07K 1/10

(52) U.S. Cl. CPC A61K 39/385; C07K 1/10; A61K 2039/627

(57) ABSTRACT

Pharmaceutical composition comprising a conjugated peptide consisting of SEQ ID NO:1 to which several peptide epitopes are grafted covalently, kit comprising the elements for manufacturing this conjugated peptide, method of synthesis and use as vaccine.

### TECHNICAL FIELD

The present invention relates to the use of the protein CRM197 (SEQ ID NO:1) as carrier protein of peptide antigens.

### PRIOR ART

Immunology is one of the most complex clinical, biological, and even biochemical fields, where a multiplicity of factors is involved in eliciting a response or controlling it.

In this context, vaccination represents a unique tool for conditioning the immune system with the desired specificity.

Various epitopes have been, and are still being, developed and used, especially peptides. These epitopes are either injected as polypeptide, for example the protein that bears them, or totally synthetic peptide epitopes are injected.

When the epitopes selected are short, they are usually coupled to a carrier protein (protéine porteuse in French), so as to ensure that they are recognized by the immune system.

These carrier proteins (carriers) are few in number.

In fact, every potential composition, and therefore every new potential carrier protein, must be tested in a suitable animal model, which is complicated and expensive and which, in addition, raises ethical questions, so that this type of study, although its aim is just the screening of new carrier proteins, is not simple. Finally, the studies, depending on whether they have been conducted on rodents or on other animals, often lead to contradictory results.

There is therefore a need for new carrier proteins.

The possible candidates include the protein CRM197. This protein is of known usage in rodents and/or when the epitope to be grafted to it is a sugar or a glycolipid, but its use for being conjugated by peptides, in particular by hydrophobic peptides, has never been implemented or in any case it has not been administered to a human patient.

Patent application WO 2016/184963 describes the grafting of short hydrophilic hexapeptides derived from the glycoprotein gp41 of the AIDS virus on protein CRM-197 or on KLH, in the hope of producing a vaccine that is effective against this disease. Crosslinking is carried out directly or by means of a heterobifunctional agent. Although several theoretical peptide/CRM-197 ratios are mentioned, no complete example describes said implementation, especially for peptides that are more complex or less hydrophilic.

Patent application EP2659906 describes an epitope of alpha-synuclein and also suggests the carrier protein CRM-197 or KLH, but does not describe coupling of peptide epitopes on CRM-197.

### BRIEF SUMMARY OF THE INVENTION

A first aspect of the present invention is a pharmaceutical composition comprising a conjugated peptide consisting of SEQ ID NO:1, to which several peptide epitopes, preferably identical, are conjugated covalently.

Advantageously, at least 3, 4, 5, 6, 7, 8, 9 or 10 peptide epitopes are grafted on a peptide of SEQ ID NO:1 (mole peptide epitope: mole SEQ ID NO:1) and/or fewer than 20, 19, 18, 17, 16, 15 peptide epitopes are conjugated to a peptide of SEQ ID NO:1 (mole peptide epitope: mole SEQ ID NO:1).

Preferably, the peptide epitopes are (each) conjugated at the level of an —NH2 residue of SEQ ID NO:1, advantageously via a heterobifunctional crosslinking agent, preferably Sulfo-GMBS.

Advantageously, a free —SH group is added to a natural peptide epitope, preferably a cysteine amino acid to the N- or C-terminal end of the peptide epitope to be grafted.

This pharmaceutical composition is advantageous for use in vaccination, preferably therapeutic, of a patient selected from the group consisting of human, dog, horse or a member of the camel family; the patient is preferably human.

Preferably, this pharmaceutical composition further comprises a vaccination adjuvant, preferably an emulsion or an oligonucleotide comprising one or more CpG units.

A related aspect of the present invention is an aqueous solution with a specified pH comprising this pharmaceutical composition and a (pH) buffer ensuring a desired pH for the solution comprising the conjugated SEQ ID NO:1.

This pH buffer is advantageously of 3.0, 3.5, 4.0, 4.5, 5.0, 5.5, 6.0, 6.5, 7.0, 7.5, 8.0 or 8.5. The above values are preferably ±0.2, even more preferably ±0.1, or even closer to the targeted value. Thus, a pH of 3.0 preferably signifies a pH greater than 2.9 and less than 3.1. The preferred pH buffers are selected from the group consisting of acetate pH 4.5, N-(2-acetamido)iminodiacetic acid pH 6.5, Bis-Tris pH 6.0, CHES (2-(N-cyclohexylamino)ethane acid) pH 9.5, citric acid pH 3.2 (or 4.0 or 5.5), imidazole pH 8, glycine pH 3.0, HEPES (4-(2-hydroxyethyl)-1-piperazine ethane sulphonic acid) pH 7.5, MES (2-(N-morpholino)ethanesulphonic acid) pH 6.2, MOPS (3-(N-morpholino)propanesulphonic acid) pH 7.0, PIPPS (piperazine-N,N′-Bis (3-propanesulphonic acid) pH 3.7 and phosphate, pH 5.0. It is to be understood that the above molecule is sometimes a salt, depending on the pH, for example the glycine pH3 buffer signifies that the glycine is in the protonated form, for example glycine-HCl.

Another related aspect of the present invention is a kit comprising an epitope derived from a natural protein comprising a free —SH group; a heterobifunctional crosslinking agent reactive for an —NH2 group and an —SH group, preferably the coupling agent Sulfo-GMBS; and a carrier protein, namely SEQ ID NO:1.

Preferably, this kit further comprises a vaccination adjuvant, preferably an emulsion or an oligonucleotide comprising one or more CpG units.

Another related aspect of the present invention is a method of coupling a peptide epitope to a carrier protein comprising the steps of identifying a peptide epitope comprising a free —SH group or of identifying a peptide epitope to which a free —SH group is added; production of the carrier protein, which is SEQ ID NO:1; activation of this carrier protein via a heterobifunctional crosslinking agent that is reactive for an —NH2 group and an —SH group so as to cause a plurality of the —NH2 groups of this SEQ ID NO:1 to react with this (hetero)bifunctional crosslinking agent; separation of the activated carrier protein from the unincorporated crosslinking agent; contacting the activated carrier protein with the identified peptide epitope so as to cause this —SH group of the peptide epitope to react with this carrier protein activated by the crosslinking agent; separation of the carrier protein coupled to several peptide epitopes from the unreacted substrates and the reaction by-products.

In this method, preferably the heterobifunctional crosslinking agent is in excess relative to the number of —NH2 residues of SEQ ID NO:1 to be activated.

Preferably, this method further comprises a step of dissolving the carrier protein (SEQ ID NO:1) coupled to several of the epitopes in an aqueous solution comprising a specified pH buffer, ensuring a desired pH for the solution comprising the conjugated SEQ ID NO:1.

This specified pH is advantageously of 3.0, 3.5, 4.0, 4.5, 5.0, 5.5, 6.0, 6.5, 7.0, 7.5, 8.0 or 8.5. The above values are preferably ±0.2, even more preferably ±0.1, or even closer to the targeted value. Thus, a pH of 3.0 preferably signifies a pH greater than 2.9 and less than 3.1.

Preferred pH buffers are selected from the group consisting of acetate pH 4.5, N-(2-acetamido)iminodiacetic acid pH 6.5, Bis-Tris pH 6.0, CHES (2-(N-cyclohexylamino)ethane acid) pH 9.5, citric acid pH 3.2 (or 4.0 or 5.5), imidazole pH 8, glycine pH 3.0, HEPES (4-(2-hydroxyethyl)-1-piperazine ethane sulphonic acid) pH 7.5, MES (2-(N-morpholino)ethanesulphonic acid) pH 6.2, MOPS (3-(N-morpholino)propanesulphonic acid) pH 7.0, PIPPS (piperazine-N,N′-Bis (3-propanesulphonic acid) pH 3.7 and phosphate, pH 5.0.

The dissolution step may advantageously be carried out, alternatively, or in synergy with the pH buffer described above, in a solution comprising acetonitrile, for example preferably said aqueous solution comprises acetonitrile, preferably between 10% and 50% (acetonitrile:water; v:v), for example, less than 40% (by volume), such as about 30:70 (acetonitrile:water; v:v). This may be coupled with a (final) step of lyophilization of the carrier protein coupled to several peptide epitopes.

Another related aspect of the present invention is a pharmaceutical composition comprising the conjugated peptide that is obtainable by the above method. Preferably, this composition further comprises a vaccination adjuvant, preferably selected from an emulsion and an oligonucleotide comprising one or more CpG units.

### DETAILED DESCRIPTION OF AN EMBODIMENT OF THE INVENTION

The protein CRM197 (SEQ ID NO:1) is doubly special in respect of the coupling of peptide epitopes owing to its high content of —NH2 residues, 40:39 lysines, i.e. almost 10% of the sequence, and the amino-terminal end.

Besides a high lysine content, SEQ ID NO:1 comprises many other charged or polar amino acids, so that the protein is naturally hydrophilic. However, when it is conjugated to several peptide epitopes, the —NH2 residues, especially the more exposed —NH2 residues, are mobilized, which upsets its hydrophilic character fundamentally. This effect is even more pronounced when the epitope to be grafted is hydrophobic, or only slightly hydrophilic.

It is certainly possible to overcome such a change in physicochemical properties, for example by selecting suitable solvents. However, the conditions must remain compatible with pharmaceutical use. Thus, (i) toxic or denaturing solvents, or (ii) conditions that are extreme or too close to the limit of solubility of SEQ ID NO:1 conjugated to several peptides, are not acceptable. Furthermore, it is convenient, during peptide synthesis, to employ a lyophilization step so as to concentrate the products of the reactions, or obtain an end product in the form of powder that is stable and easy to transport; however, certain organic solvents, such as acetonitrile, cannot be used at excessively high concentrations in these conditions as this involves risks of explosion.

A first aspect of the present invention is therefore a pharmaceutical composition comprising a conjugated peptide consisting of SEQ ID NO:1 to which several peptide epitopes are grafted covalently. This composition is preferably for pharmaceutical use (vaccination).

In the context of the present invention, the terms “vaccine” or “vaccination” are preferably understood in their broadest sense meaning any injection of a peptide epitope in a patient with the aim of eliciting a specific response of the immune system. Thus, the terms “vaccine” or “vaccination”, in the context of the present invention, advantageously include any immunization of a patient. The desired aim may be preventive or therapeutic. Immunization (vaccination) is not limited to infectious diseases but also includes inflammatory, autoimmune, and degenerative diseases, including cancer. The response may be humoral or cellular, or both. It may be a (specific) activation of the immune system or a (specific) repression of the immune system. The type of cell of the immune system targeted is not limited, nor is the nature of the antigen-presenting cell.

Preferably, the peptide epitopes to be grafted on a molecule of SEQ ID NO:1 are identical, although grafting of several different epitopes could very well be carried out on one molecule of SEQ ID NO:1.

Advantageously at least 3, 4, 5, 6, 7, 8, 9 or 10 peptide epitopes, but preferably fewer than 20, 19, 18, 17, 16, 15 peptide epitopes are grafted on a peptide of SEQ ID NO:1 (moles peptide epitopes:moles SEQ ID NO:1).

A precise and/or predictable number of peptide epitopes per SEQ ID NO:1 allows purification of the grafted protein to be carried out better.

A high number of peptide epitopes per SEQ ID NO:1 is difficult to obtain, especially for hydrophobic peptides, but allows easier separation, and greater administration of the peptide epitopes to a patient, which is particularly advantageous in therapeutic vaccination.

Advantageously, the peptide epitopes are grafted at the level of the —NH2 residues of SEQ ID NO:1, sometimes via a “spacer”, such as the remainder of the coupling agent, depending on the coupling agent used (e.g. Sulfo-GMBS).

Even if grafting (conjugation) transforms a charged residue, very hydrophilic and exposed, into a hydrophobic complex, which greatly increases the hydrophobic character of the resultant complex especially when several epitopes have been grafted, the inventors succeeded in identifying conditions in which peptide epitopes to be grafted may be only slightly hydrophilic, or even hydrophobic.

In the context of the present invention, “peptide epitope” means any molecule formed essentially by amide bonds, such as natural peptides, made up of natural amino acids. However, the peptide epitopes may comprise modified amino acids: either modifications that occur in certain pathological or physiological situations (e.g. at the level of proline, serine, asparagine, lysine, arginine or tryptophan) or other modifications of a chemical type, such as coupling of an amino acid by an alkyl residue.

Preferably, in the context of the present invention, a peptide epitope comprises at least one chain of 7 amino acids linked by peptide bonds, preferably at least 8, 9, 10, 11, 12, 13, 14 and/or preferably less than 40 amino acids, less than 35, less than 30, or even less than 20 amino acids.

These peptide epitopes are therefore grafted covalently to SEQ ID NO:1; in practice several peptide epitopes are grafted to it. These peptide epitopes are also incorporated in the kit comprising the heterobifunctional crosslinking agent that is reactive for an —NH2 group and an —SH group, and are used in the corresponding coupling process, and are therefore incorporated in the pharmaceutical composition obtainable by this method.

In the context of the present invention, “peptide that is only slightly hydrophilic or is hydrophobic” means a peptide that comprises less than 50%, preferably less than 40%, less than 35%, or even less than 30%, or even less than 26% of amino acids selected from the group consisting of arginine, lysine (and/or ornithine, citrulline), histidine, glutamic acid, aspartic acid and/or more than 20%, 30%, 35%, or even 40% of amino acids selected from the group consisting of valine, leucine, isoleucine (and/or norleucine), methionine, proline, phenylalanine, tyrosine and tryptophan. Preferably, a hydrophobic peptide comprises, in the context of the present invention, at least 35% of amino acids selected from the group consisting of valine, leucine, isoleucine (and/or norleucine), methionine, proline, phenylalanine, tyrosine and tryptophan and less than 35% of amino acids selected from the group consisting of arginine, lysine (and/or ornithine, citrulline), histidine, glutamic acid and aspartic acid. More generally, in the context of the present invention, preferably a hydrophobic peptide comprises more (e.g. 1, 2, 3, 4, 5, 6, 7, 8, or more) amino acids selected from the group consisting of valine, leucine, isoleucine (and/or norleucine), methionine, proline, phenylalanine, tyrosine and tryptophan than amino acids selected from the group consisting of arginine, lysine (and/or ornithine, citrulline), histidine, glutamic acid, aspartic acid and, potentially, serine. Other non-natural amino acids, which includes natural or artificial modifications of the side chains of the amino acids, also have hydrophilic or hydrophobic properties. The consequences with regard to the hydrophobic character of the peptide resulting from their possible incorporation in a peptide epitope will be found for example by adapting the above numerical values.

Alternatively, the terminology “peptide that is only slightly hydrophilic” relates to peptides with low solubility in water or in aqueous solutions (without detergent), for example at pH 7. This may be determined from their distribution in a water (pH 7; 25° C.)—octanol system: a peptide that is only slightly hydrophilic being found predominantly (e.g. more than 50%, more than 60%, more than 70%, more than 80%, more than 90%, or even more than 95%) in the organic phase (e.g. octanol).

The present invention therefore advantageously allows the grafting of peptides (peptide epitopes), even of hydrophobic peptides, in large amount on CRM-197. The grafting of hydrophobic (or only slightly hydrophilic) peptides is therefore possible and even advantageous.

Preferably, the epitopes are grafted via a heterobifunctional crosslinking agent, preferably reactive for an —NH2 group and an —SH group, even more preferably Sulfo-GMBS.

In the case when a crosslinking agent that is reactive for an —SH group is used, an (accessible) —SH group is advantageously added to a peptide epitope that would be naturally devoid of it (or whose —SH groups would be inaccessible). A preferred manner is the coupling (grafting) of a cysteine residue to a carboxy- or amino-terminal end) of the epitope. This cysteine that is optionally added is preferably incorporated in the above calculation for estimating the hydrophobic character of the peptide to be grafted.

In the case when the peptide epitope to be coupled (grafted) contains a cysteine naturally, preferably the coupling (grafting) step comprises a preliminary step for determining whether this cysteine is accessible for coupling (grafting) and, advantageously, whether coupling (grafting) on this cysteine changes the immunogenic character of the peptide epitope. Thus, preferably, in the case when the peptide epitope contains a cysteine naturally (accessible or not; problematic if used for coupling or not), this cysteine may be protected, and another cysteine residue may be added to one of the ends of the peptide epitope.

This pharmaceutical composition is advantageous for use in vaccination of a patient selected from the group consisting of human (Homo sapiens), dog (Canis vulgaris), horse (Equus caballus) and a member of the camel family (Camelus sp.); preferably the patient is human. In fact, the large number of peptides coupled to SEQ ID NO:1 allows massive administration of the epitopes to the patient, which is particularly advantageous in therapeutic vaccination.

This therapeutic vaccination is useful for treating infectious diseases especially when the infectious agent is intracellular, autoimmune diseases, inflammatory diseases, degenerative diseases and cancer.

Advantageously, the pharmaceutical composition is used in the presence of, or is used in combination with, a (vaccination and/or immunization) adjuvant, such as emulsions (oil-in-water and water-in-oil), oligonucleotides having CpG units or aluminium salts. The preferred adjuvants are emulsions (oil-in-water and water-in-oil) and oligonucleotides having CpG units.

Preferably, the pharmaceutical composition is, or will be with a view to its administration to the patient, in aqueous solution in a buffer ensuring a desired pH for the solution comprising the conjugated SEQ ID NO:1. This pH buffer is advantageously of 3.0, 3.5, 4.0, 4.5, 5.0, 5.5, 6.0, 6.5, 7.0, 7.5, 8.0 or 8.5. The above values are preferably ±0.2, even more preferably ±0.1, or even closer to the targeted value. Thus, a pH of 3.0 preferably signifies a pH greater than 2.9 and less than 3.1.

The preferred pH buffers are selected from the group consisting of acetate pH 4.5, N-(2-acetamido)iminodiacetic acid pH 6.5, Bis-Tris pH 6.0, CHES (2-(N-cyclohexylamino)ethane acid) pH 9.5, citric acid pH 3.2 (or 4.0 or 5.5), imidazole pH 8, glycine pH 3.0 (i.e. glycine-HCl), HEPES (4-(2-hydroxyethyl)-1-piperazine ethane sulphonic acid) pH 7.5, MES (2-(N-morpholino)ethanesulphonic acid) pH 6.2, MOPS (3-(N-morpholino)propanesulphonic acid) pH 7.0, PIPPS (piperazine-N,N′-Bis (3-propanesulphonic acid) pH 3.7 and phosphate, pH 5.0. Preferably, these pH buffers are used at a concentration between 5 and 50 mM, preferably between 20 and 50 mM. The pH values are preferably within a range of ±0.2; thus “pH 5.0” signifies from 4.8 to 5.2, even if a pH of strictly 5.0 is preferred (in this example explaining the pH range). Advantageously an additive is combined with the pH buffer, preferably selected from arginine and glutamine (50 mM each), trimethylamine N-oxide (500 mM), Tween®20 (1%; w:v), trehalose (500 mM) and glycerol (20%; v:v).

An associated aspect of the present invention is a kit comprising one or more epitopes derived from a natural protein comprising a free —SH group; a heterobifunctional crosslinking agent that is reactive for an —NH2 group and an —SH group, preferably the coupling agent Sulfo-GMBS; and SEQ ID NO:1. This kit contains the essential elements for coupling one or more epitopes to SEQ ID NO:1.

Advantageously, this kit also comprises the adjuvant described above and/or the pH buffer described above.

Another associated aspect of the present invention is the method of coupling a peptide epitope to a carrier protein comprising the steps of:
identifying a peptide epitope comprising a free —SH group or identifying a peptide epitope to which a free —SH group is added (e.g. a cysteine to the amino-terminal end);
obtaining the carrier protein, which is SEQ ID NO:1;
activating the carrier protein by means of a heterobifunctional crosslinking agent that is reactive for an —NH2 group and an —SH group so as to cause a plurality of —NH2 groups (of this SEQ ID NO:1) to react with the bifunctional crosslinking agent;
separating the activated carrier protein from the unincorporated crosslinking agent;
contacting the activated carrier protein with the peptide epitope identified so as to cause the —SH group of the peptide epitope to react with the carrier protein activated by the crosslinking agent;
optionally, adding an excess of cysteine so as to neutralize the reactive residues of the —SH groups that would not have reacted with the epitope added (in excess);
separating the carrier protein coupled to several peptide epitopes of the unreacted substrates and of the reaction by-products;
optionally, putting the carrier protein coupled to several peptide epitopes in an aqueous solution comprising a specified pH buffer, ensuring a desired pH for the solution comprising the conjugated SEQ ID NO:1;
optionally, lyophilizing the carrier protein coupled to several peptide epitopes;
optionally, redissolving the carrier protein in an aqueous solution comprising the pH buffer described above.

Preferably, just as for the other aspects of the present invention, these pH buffers are used at a concentration from 5 to 50 mM, preferably from 20 to 50 mM. This pH buffer is advantageously of 3.0, 3.5, 4.0, 4.5, 5.0, 5.5, 6.0, 6.5, 7.0, 7.5, 8.0 or 8.5. The above values are preferably ±0.2, even more preferably ±0.1, or even closer to the targeted value. Thus, a pH of 3.0 preferably signifies a pH greater than 2.9 and less than 3.1. Preferred pH buffers are selected from the group consisting of acetate pH 4.5, N-(2-acetamido)iminodiacetic acid pH 6.5, Bis-Tris pH 6.0, CHES (2-(N-cyclohexylamino)ethane acid) pH 9.5, citric acid pH 3.2 (or 4.0 or 5.5), imidazole pH 8, glycine pH 3.0 (glycine-HCl), HEPES (4-(2-hydroxyethyl)-1-piperazine ethane sulphonic acid) pH 7.5, MES (2-(N-morpholino)ethanesulphonic acid) pH 6.2, MOPS (3-(N-morpholino)propanesulphonic acid) pH 7.0, PIPPS (piperazine-N,N′-Bis (3-propanesulphonic acid) pH 3.7 and phosphate, pH 5.0. The pH values are preferably within a range of ±0.2; thus “pH 5.0” signifies from 4.8 to 5.2, even if a pH of strictly 5.0 is preferred (in this example explaining the pH range). Advantageously, an additive is combined with the pH buffer, preferably selected from arginine and glutamine (50 mM each), trimethylamine N-oxide (500 mM), Tween®20 (1%; w:v), trehalose (500 mM) and glycerol (20%; v:v).

The inventors noticed that the activation of the —NH2 groups of SEQ ID NO:1, followed sequentially by grafting of the epitopes (e.g. via a free and/or accessible —SH residue), allows a larger and more reproducible charge. A “valence” of 12 to 13 grafted epitopes (on average) on a molecule of SEQ ID NO:1 was obtained reproducibly for at least two different epitopes, despite their hydrophobicity (a very hydrophobic epitope and a moderately hydrophobic epitope).

The inventors noticed that a water:acetonitrile mixture 70:30 (v:v) made it possible to dissolve grafted SEQ ID NO:1, even when several (2, 3, 4, 5, 6, 7, 8, 9, 10, 11, 12, or even 13) hydrophobic epitopes were grafted on a molecule of SEQ ID NO:1. Moreover, incorporation of the pH buffer described above in an aqueous solution ensures good solubility of the conjugated carrier protein. Preferably, in this method, the heterobifunctional crosslinking agent is in excess relative to the number of —NH2 residues of SEQ ID NO:1 to be activated, which allows maximum activation of the exposed —NH2 groups.

Also preferably, the epitope is in (slight, e.g. less than 3-fold) excess relative to the number of —NH2 residues of SEQ ID NO:1; once again, this makes it possible to ensure maximum, constant grafting.

Preferably, the conjugated SEQ ID NO:1 is maintained in solution in the presence of acetonitrile at a content below 40% (v:v), ideally at about 30% (v:v). Alternatively, incorporation of the pH buffer described above in an aqueous solution ensures good solubility for the conjugated carrier protein (SEQ ID NO:1). This makes it possible to carry out a final step of lyophilization of the conjugated product.

The lyophilization step is particularly advantageous when organic solvents (e.g. acetonitrile) have been used, at concentrations allowing lyophilization.

Alternatively, the conjugated SEQ ID NO:1 may be solubilized in the absence of organic solvents, for example using the buffer mixture described above. In this case, the lyophilization step is not preferred, but optional.

These various finishing steps are useful for a pharmaceutical composition comprising this peptide conjugated to SEQ ID NO:1. This (lyophilized) composition is stable, easily transportable and allows long-term storage.

### EXAMPLES

Of course, the present invention is not in any way limited to the embodiments described above, and many modifications may be made to it while remaining within the scope of the accompanying claims.

### Example 1: Synthesis of SEQ ID NO:1 and of the Peptides of SEQ ID NO:2-4

SEQ ID NO:1 was obtained by fermentation of a particular strain of Escherichia coli; the sequence was modified so that the protein (mature, SEQ ID NO:1) is secreted in the periplasmic space, for example by the method described in patent application BE2021/5137 filed on 26 Feb. 2021. The protein is recovered and purified by filtration and by chromatography, for example by the method described in patent application BE2021/5138 filed on 26 Feb. 2021. None of the reagents used is of animal origin, and the content of residual endotoxins is compatible with clinical use.

The peptides of SEQ ID NO:2, 3, 4 and 5 were obtained by solid phase synthesis. However, other methods of synthesis, or even of fermentation, are possible. In practice, the C-terminal end of the peptide is fixed to a polymer support and the peptide chain is constructed and then extended to the N end by repeating coupling cycles; the carboxy end of the amino acid to be incorporated being activated, and then coupled to the amino end of the peptide fixed to the resin. The amino group is protected by the Fmoc group and the other potentially reactive functional groups are protected by other groups. When the amino acid is grafted on the peptide to be extended, after removal of the amino acids in excess and the washing steps, the Fmoc group is cleaved by adding piperidine. Cleavage of the resin and deprotection of the functional groups of the side chains of the amino acids is carried out by adding trifluoroacetic acid (TFA).

In certain cases, the tryptophan residue of SEQ ID NO:2 or of SEQ ID NO:4 is alkylated on the indole group by adding an amide-Tmob to TFA. Moreover, other modifications are possible.

After cleavage of the resin, deprotection, purification (e.g. reversed-phase HPLC) and filtration (0.45 μm), the peptides were analysed by mass spectrometry (MALDI-TOF) and by HPLC. The steps in HPLC are carried out each time by means of a mixture of water (0.1% TFA) with an acetonitrile gradient (0.1% TFA), and detection by UV (215 and 280 nm). The purity must be greater than 90%, or even 95%. In practice, the inventors obtained peptides with a purity of 97%.

### Example 2: Coupling by Means of EDC, Relative to Glutaraldehyde

The inventors first tried to carry out coupling using the method described in patent EP2004217, but applied to peptides of sequences SEQ ID NO:2 and SEQ ID NO:4 to be coupled on SEQ ID NO:1 instead of KLH.

This type of coupling works, but is not satisfactory in practice. In fact, the inventors observed that the molecule obtained was difficult to purify or to sterilize by filtration and there was lack of uniformity from batch to batch. One of the explanations advanced by the inventors is that the hydrophilic character of the unmodified protein is transformed excessively when a peptide is grafted thereon multiple times, which makes this carrier protein unusable for the downstream treatments. Thus, the protein of SEQ ID NO:1 is useful as a carrier, but this usefulness is not evident in the context of the grafting of peptide epitopes, in particular of hydrophobic peptide epitopes.

However, the inventors nevertheless tried to overcome this difficulty by choosing another coupling agent, 1-ethyl-3-[3-dimethylaminopropyl] carbodiimide hydrochloride (EDC); available for example from Pierce. This is an agent that allows coupling of carboxyl groups to primary amines. EDC reacts with the carboxyl group to form a reactive intermediate, O-acylisourea. Although this intermediate does not react with an amine, it undergoes hydrolysis, regenerating the carboxyl group.

For this purpose, the inventors developed and compared a one-step method of coupling, and a two-step method of coupling.

In the one-step method, like the method based on glutaraldehyde, the carrier protein (SEQ ID NO:1), EDC and the epitope of SEQ ID NO:2 or that of SEQ ID NO:4 are brought together in the same vessel with magnetic stirring, and then the precipitate is recovered and purified on Sephadex™ G50 prior to lyophilization.

In the two-step method, SEQ ID NO:1 is activated firstly by being put in contact with EDC, then the epitope of SEQ ID NO:2 or that of SEQ ID NO:4 is added prior to separation, purification and lyophilization.

With respect to glutaraldehyde, the reaction product is usable, even if there are still unusable aggregates, synonymous with losses of yield, and a significant part of SEQ ID NO:1 remains in solution, synonymous with unreacted (or insufficiently reacted) protein, i.e. a second loss of yield. Moreover, the efficacy of coupling varies considerably from batch to batch, and the solubility of the conjugate is mediocre, or even insufficient, which further complicates sterilization by filtration.

The yield was mediocre in the case of the one-step method: 12% for grafting of SEQ ID NO:2 and 30% for grafting of SEQ ID NO:4. However, this yield increases, for example to 60% when the coupling process is carried out in two steps (SEQ ID NO:1 with SEQ ID NO:4). The molar distribution ratio, SEQ ID NO:1 vs SEQ ID NO:2 or SEQ ID NO:4, is less than 2 for the one-step method of coupling, and is greater than 2 for the 2-step method of coupling.

### Example 3. Coupling of Protein CRM197 with a Peptide by Means of GMBS

The inventors dissolved 25 mg of carrier protein CRM197 (SEQ ID NO:1) in 2.5 ml of an aqueous solution. The carrier protein was from a batch compatible with clinical use and had a content of endotoxins below the limit of detection.

The inventors weighed 19 mg of Sulfo-GMBS (Pierce; reference 22324; N-γ-maleimidobutyryl-oxysulphosuccinimide ester), and then added 2.5 ml of a conjugation medium (100 mM of “Phosphate-Buffered Saline”, PBS and 10 mM of ethylenediamine tetraacetic acid, EDTA) and 2.5 ml of the solution of SEQ ID NO:1. This mixture is stirred with a magnetic stirrer for 1 h at 4° C.

The inventors purified SEQ ID NO:1 activated by passage on a Sephadex™ G50 column equilibrated with PBS buffer, pH 7.2, monitoring the recovery of SEQ ID NO:1 at 280 nm.

The inventors transferred the fraction(s) containing the absorption peak at 280 nm (the protein CRM-GMBS) to the bottle containing SEQ ID NO:3 (25 mg, dissolved in DMSO), which was then stirred with a magnetic stirrer for 2 h at 4° C. The conjugate that forms between SEQ ID NO:1 and SEQ ID NO:3 precipitates. After centrifugation, the pellet is dissolved in acetonitrile.

The inventors added 1 ml of 1M cysteine, stirring for 30 minutes.

The inventors transferred the reaction mixture to a 50 ml Falcon® tube; it is centrifuged and the pellet is separated and taken up in a water:acetonitrile mixture (70:30 w:w).

The inventors carried out purification on a Sephadex™ G50 column, again monitoring absorption at 280 nm.

The inventors lyophilized the purified conjugate.

All of the above steps were carried out in conditions compatible with later clinical use. Studies of stability with a view to clinical use were then carried out. The powder stored at a temperature of −20° C. and of +5° C. remains stable for several months. It withstands accelerated ageing (stress at 25° C.) for at least 14 days (measurements by unmodified SDS-PAGE, MALDI-TOF, capillary electrophoresis and infrared (FTIR) for evaluating any degradation of chemical residues or of secondary structure).

The analysis reveals that SEQ ID NO:1 reacted very predominantly with the peptide of SEQ ID NO:3; a yield of 64% was obtained. Besides a good yield, the molar ratio of SEQ ID NO:1 to SEQ ID NO:3 is greater than 8. Values of 13 were even found, and were obtained reproducibly for this coupling yield. Even higher ratios were obtained, but at the expense of a lower coupling yield (e.g. 36 and 47%).

In practice, the method of coupling with heterobifunctional agents, especially when it is carried out in two steps, even allows a level of coupling of more than 50 μg of peptide on 100 μg of SEQ ID NO:1.

SEQ ID NO:1 that has incorporated several peptides of SEQ ID NO:3 or 5 (see below) is easily differentiated (e.g. by electrophoresis) since its molecular weight changes from 58 kDa to ˜78 KDa if 10 peptides are grafted on one molecule of SED ID NO:1, or even ˜90 kDa if more peptides are grafted on one molecule of SEQ ID NO:1; e.g. 13 or 14 peptides grafted on one molecule.

### Example 4

The same protocol is applied for the peptide of SEQ ID NO:5. The coupling yield is also excellent, just like the number of peptides coupled to SEQ ID NO:1.

### Example 5

The peptide from example 3 (182 μg, equivalent to 60 μg of SEQ ID NO:3) is combined with the peptide from example 4 (182 μg, equivalent to 60 μg of SEQ ID NO:5). An adjuvant is added to this mixture or to a placebo. The active ingredient or the placebo is injected in a cohort of human patients with myasthenia gravis (MG) in double-blind conditions. In practice, 3 sequential injections are carried out (week 1, 5 and 13). Then the same protocol is designed for a second cohort of patients, but with even more active ingredient. Finally, the study is continued as an “open label” study, to evaluate the long-term tolerability and efficacy of the treatment. At each injection, the patient is closely monitored for any side-effects, firstly in the hospital with close supervision, and then at home. Blood samples are taken for immunogenicity assays.

The data generated make it possible to conclude that there is good tolerability (the active ingredient does not cause more side-effects than the placebo) and efficacy of the treatment.

Thus, surprisingly, the inventors demonstrated that SEQ ID NO:1 is a good carrier protein for coupling peptide epitopes with a view to therapeutic use.

1. A pharmaceutical composition comprising a conjugated peptide consisting of SEQ ID NO:1 to which a plurality of peptide epitopes have been grafted covalently, in which each of said plurality of peptide epitopes comprises a chain of at least 7 amino acids linked by peptide bonds.

2. The pharmaceutical composition according to claim 1, in which the plurality of peptide epitopes are identical.

3. The pharmaceutical composition according to claim 1, in which the plurality of peptide epitopes comprises at least peptide epitopes grafted on the peptide of SEQ ID NO:1 (mole peptide epitope:mole SEQ ID NO:1).

4. The pharmaceutical composition according to claim 1, in which the plurality of peptide epitopes comprises fewer than 20 peptide epitopes grafted on the peptide of SEQ ID NO:1 (mole peptide epitope:mole SEQ ID NO:1).

5. The pharmaceutical composition according to claim 1, in which each of the plurality of peptide epitopes is grafted at the level of an —NH2 residue of SEQ ID NO:1.

6. The pharmaceutical composition according to claim 1, in which the plurality of peptide epitopes are grafted via a heterobifunctional crosslinking agent.

7. The pharmaceutical composition according to claim 6, in which the heterobifunctional crosslinking agent is reactive for an —NH2 group and an —SH group.

8. The pharmaceutical composition according to claim 7, in which either at least one of the plurality of peptide epitopes comprises a free —SH group, or a free —SH group is added to a natural peptide epitope of the plurality of peptide epitopes.

9. The pharmaceutical composition according to claim 8, in which a cysteine residue is grafted to the amino- or carboxy-terminal end of at least one of the plurality of peptide epitopes.

10. A method for immunization of a patient, the method comprising administering the pharmaceutical composition of claim 1 to the patient, the patient being selected from the group consisting of a human, a dog, a horse, or a member of the camel family.

11. The method for immunization according to claim 10, in which the immunization is for treating a condition selected from the group consisting of infectious diseases, autoimmune diseases, inflammatory diseases, degenerative diseases, and cancers.

12. The pharmaceutical composition according to claim 1, further comprising a vaccination adjuvant.

13. An aqueous solution comprising the pharmaceutical composition according to claim 1, said aqueous solution comprising a buffer so as to ensure a specified pH for said aqueous solution.

14. A kit comprising:
an epitope derived from a natural protein comprising a free —SH group;
a heterobifunctional crosslinking agent that is reactive for an —NH2 group and an —SH;
SEQ ID NO:1,
in which said epitope derived from a natural protein comprises a chain of at least 7 amino acids linked by peptide bonds.

15. The kit according to claim 14, further comprising a vaccination adjuvant.

16. A method of coupling a peptide epitope to a carrier protein comprising the steps of:
identifying a peptide epitope comprising a free —SH group and/or identifying a peptide epitope to which a free —SH group is added;
obtaining the carrier protein, which is SEQ ID NO:1;
activating said carrier protein by means of a heterobifunctional crosslinking agent that is reactive for an —NH2 group and an —SH group so as to cause a plurality of —NH2 groups of said SEQ ID NO:1 to react with said heterobifunctional crosslinking agent;
separating the activated carrier protein from unincorporated crosslinking agent;
contacting said activated carrier protein with said peptide epitope identified so as to cause said —SH group of said peptide epitope to react with said activated carrier protein;
separating the activated carrier protein coupled to a plurality of said peptide epitopes from unreacted substrates and of the from reaction by-products,
in which said peptide epitope comprises a chain of at least 7 amino acids linked by peptide bonds.

17. The method according to claim 16, in which the heterobifunctional crosslinking agent is in excess relative to the number of —NH2 residues of SEQ ID NO:1 to be activated.

18. The method according to claim 16, further comprising a step of dissolving the activated carrier protein coupled to the plurality of the epitopes in an aqueous solution comprising a buffer so as to ensure a specified pH for said aqueous solution.

19. The method according to claim 16, further comprising a step of lyophilization of the activated carrier protein coupled to the plurality of peptide epitopes.

20. A pharmaceutical composition comprising the conjugated peptide that is obtainable by the method according to claim 16.

21. The pharmaceutical composition according to claim 20, further comprising a vaccination adjuvant.

22. The pharmaceutical composition according to claim 1, in which the plurality of peptide epitopes are hydrophobic.

23. The method according to claim 18, in which said aqueous solution comprises acetonitrile.

24. The method according to claim 18, in which said aqueous solution comprises about 10 vol % to about 50 vol % acetonitrile.
